(19) United States
(12) Patent Application Publication
(10) Pub. No.: US 2026/0017540 A1
(43) Pub. Date: Jan. 15, 2026

(54) ADAPTIVE AI GOVERNANCE AND INFERENCE SYSTEM FOR PROACTIVE TASK EXECUTION

(71) Applicant: ALESHA LYNN ARP, COTO DE CAZA, CA (US)

(72) Inventor: ALESHA LYNN ARP, COTO DE CAZA, CA (US)

(21) Appl. No.: 19/192,084

(22) Filed: Apr. 28, 2025

Related U.S. Application Data
(60) Provisional application No. 63/671,203, filed on Jul. 13, 2024.

(51) Int. Cl. G06N 5/04; H04L 9/00

(52) U.S. Cl. CPC G06N 5/04; H04L 9/008

(57) ABSTRACT

A device, method, and system that integrates rule-based governance with AI-driven inferencing to optimize decision-making, enhance privacy, and conduct preemptive actions in various domains such as healthcare, compliance, and personal safety. The device, method and system comprises data collection interfaces, a Content Control Module (CCM), an Inference Agent Controller (IAC), Rules Controller (RC) utilizing Virtual Experts, and Action Agents that execute decisions. The invention dynamically adapts to changing contexts, ensuring real-time intervention and enhanced accuracy.

### PRIORITY CLAIM

This application claims the benefit of priority under 35 U.S.C. § 119(e) to U.S. Provisional Patent Application No. 63/671,203, filed on Jul. 13, 2024, titled “SITUATIONALLY AWARE AUTONOMOUS METHOD, SYSTEM AND DEVICE WITH RULE-BASED PREEMPTIVE DECISIONING CAPABILITIES”, which is incorporated herein by reference in its entirety.

### FIELD OF THE INVENTION

This invention relates to artificial intelligence (AI)-powered expert systems and decision-making frameworks, specifically those that utilize rule-based governance and machine learning inference to provide proactive, anticipatory assistance in various domains, including healthcare, compliance, security, and personal wellness.

### BACKGROUND OF THE INVENTION

Conventional AI-driven systems often require user prompting and manual interaction to generate relevant outputs. Additionally, these systems lack robust mechanisms for privacy, real-time adaptation, and preemptive decision-making. Existing solutions provide either probabilistic inference without structured governance or rigid rule-based systems incapable of adaptive learning. There exists a need for an integrated AI expert system that dynamically adapts based on real-time context and applies domain-specific governance rules to enhance decision accuracy and user experience.

### SUMMARY OF THE INVENTION

Accordingly, the disclosed invention is a method, system, and device that integrates AI-driven inferencing agents with rule-based governance to conduct preemptive decision-making in real-time. This invention applies expert-defined rules to contextual data and autonomously selects inference engines and Action Agents to deliver optimized, privacy-preserving, and contextually relevant results. The system dynamically adapts rules and virtual expert profiles based on evolving data and situational changes.

The system disclosed herein is designed to conduct preemptive actions using artificial intelligence and rule-based governance, integrating a combination of data collection interfaces, Inference Agent Controllers (IAC), a Rules Controller (RC), a Content Control Module (CCM), and Action Agents. The data collection interfaces are configured to receive inputs containing both real-time and historical situational information, ensuring a comprehensive contextual understanding of the operating environment. These interfaces seamlessly interact with various sensors and external data sources to enhance the accuracy and reliability of inferencing operations.

At the core of the system, the Inference Agent Controller (IAC) is responsible for managing and orchestrating multiple Inference Agents that analyze incoming inputs and derive meaningful contextual insights. These Inference Agents employ advanced machine learning models and probabilistic reasoning techniques to refine decision-making accuracy and cross-validate inferencing outputs. The Rules Controller operates in conjunction with the Inference Agent Controller (IAC), ensuring the enforcement and continuous adaptation of domain-specific governance rules. By dynamically updating these rules in response to evolving environmental factors and user interactions, the system achieves a high degree of operational flexibility and responsiveness.

The Content Control Module (CCM) plays a critical role in regulating the amount and type of input data processed by the system. Through an intelligent filtering mechanism, it ensures that only relevant and authorized data is utilized for inferencing while maintaining compliance with stringent privacy and governance standards. This module incorporates advanced encryption techniques and anonymization protocols to preserve user confidentiality and prevent unauthorized access to sensitive information.

Upon completion of the inferencing process, Action Agents execute the system's recommended tasks, leveraging real-time contextual data and pre-established domain-specific rules. These agents interact with external devices and automation frameworks to implement proactive interventions, such as adjusting system parameters, notifying users of potential risks, or autonomously executing corrective actions. By integrating these components into a cohesive framework, the system effectively delivers real-time, AI-driven decision-making capabilities while ensuring adherence to regulatory and ethical guidelines.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a block diagram illustrating the interaction between rule-based governance, Inference Agents, and Action Agents;

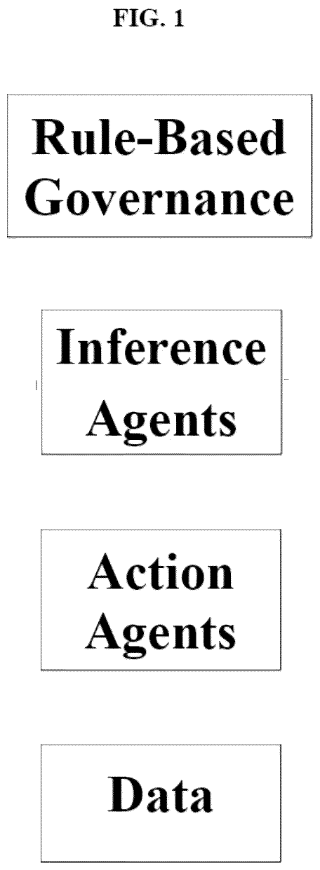

FIG. 2 illustrates a diagram depicting how real-time and historical data are processed to generate insights; and

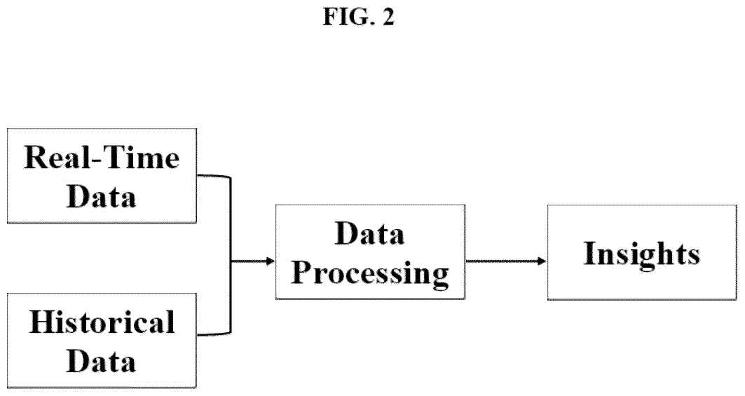

FIG. 3 illustrates a flowchart showing how the system assists a user in making health-related decisions based on dietary, physical activity, and biometric data.

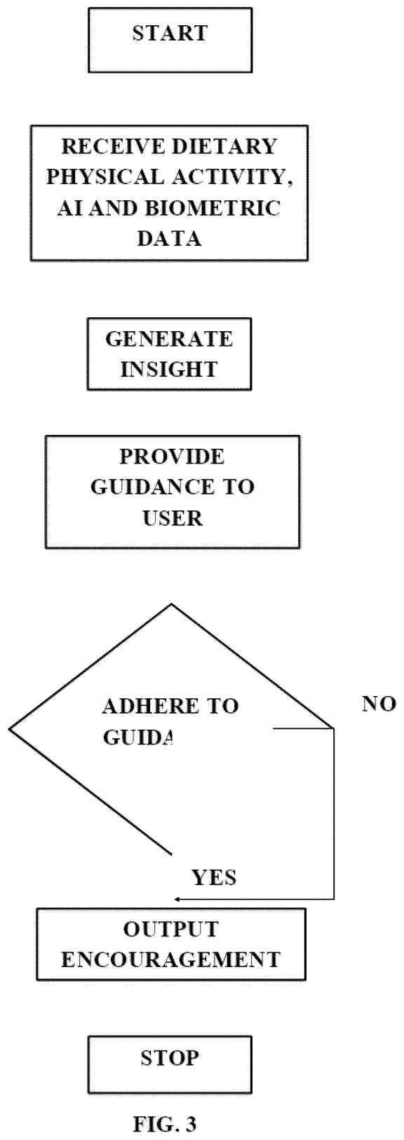

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a”, “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can”, “could”, “might”, or “may”, unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

The system is designed to facilitate intelligent, context-aware decision-making by leveraging a combination of AI-based Inference Agents, rule-based governance controllers, and Action Agents. The architecture consists of multiple interdependent components that operate in coordination to achieve situational awareness, inferencing, and task execution.

The system collects data from multiple sources to create a holistic understanding of the user's environment and context. These data sources include automated sensors, user input modules, and external integration.

The automated sensors are devices such as biometric scanners, environmental sensors, accelerometers, and gyroscopes that may continuously capture physiological, behavioral, and environmental data. Via the Input Interface, (data collection interfaces) users can manually enter data using voice commands, text input, or image recognition. External integration provides the system interfaces with wearable health trackers, medical diagnostic tools, smart home devices, and other IoT-enabled systems to enrich its knowledge base.

The Content Control Module governs the way data is ingested, processed, and utilized within the system. It ensures compliance with privacy and security standards while optimizing the inferencing process by privacy filtering, data governance, and dynamic data handling.

Privacy filtering provides for the removal or anonymizing of personally identifiable information (PII) before data is fed into inference engines. Data governance applies access control rules and regulatory policies (e.g., HIPAA, GDPR) to filter sensitive data. Dynamic data handling adjusts data granularity based on user preferences, system confidence levels, and context-specific requirements.

At the core of the system is a robust Rules Controller (RC) that governs decision-making through pre-defined and dynamically generated rules. The RC consists of Virtual Experts, real-time rule adaptation and domain-specific governance. Virtual Experts herein provide pre-configured expert knowledge modules that encapsulate domain-specific rule sets. In real-time, the system continuously evaluates user behavior and environmental changes, updating active rule sets accordingly. Domain-specific governance provides customizable governance frameworks that enable sector-specific applications, such as medical decision support or compliance monitoring.

The Inference Agent Controller (IAC) serves as the orchestrator for AI-driven inferencing tasks. It manages multiple inference engines to ensure accuracy, consistency, and reliability in contextual understanding. IAC provides for multi-agent coordination, confidence scoring and cross-validation. Multi-agent coordination distributes inferencing tasks among specialized AI agents, such as computer vision modules, natural language processing (NLP) engines, and statistical pattern analyzers. Confidence scoring implements probabilistic models to validate inference outputs and flag uncertain results for further evaluation. Cross-validation combines insights from multiple inference engines to refine predictions and enhance reliability.

Once inferencing is complete, Action Agents execute tasks based on the processed insights. These Agents issue real-time recommendations, which notifies users of health risks, regulatory compliance issues, or security threats; automates corrective actions, which adjusts system parameters, modifies security settings, or controls smart devices based on inferred recommendations; and facilitates user interactions, which provides interactive feedback mechanisms, such as chatbot-driven guidance or personalized alerts.

The system can be integrated into healthcare ecosystems to provide real-time decision support by monitoring chronic conditions through continuous glucose, heart rate, and activity tracking; providing tailored dietary recommendations based on individual metabolic profiles and meal composition; and alerting healthcare providers when anomalous biometric patterns are detected.

The system ensures regulatory compliance and data privacy by filtering sensitive information before AI processing; enforcing HIPAA, GDPR, and other regulatory frameworks through rule-based data governance; and generating compliance audit reports for businesses utilizing AI-powered data analysis.

The system herein can identify and mitigate behavioral risks in high-stakes environments such as industrial safety and security monitoring. It is useful for workplace safety enforcement and ensures adherence to safety protocols by monitoring user activities and triggering interventions when risky behaviors are detected. It also provides personal security assistance by use of computer vision and real-time location tracking to detect distress scenarios and autonomously notify emergency responders.

The implementation of this system requires an advanced hardware infrastructure capable of supporting high-speed data processing, real-time inferencing, and secure governance rule enforcement. The primary hardware components consist of high-performance computing units, specialized inference accelerators, storage solutions, and advanced sensors for data collection.

The data collection interfaces demand an array of sophisticated sensors and IoT devices to gather real-time situational data. This includes OMRON® healthcare sensors, GARMIN® smartwatches, and ABBOTT® FreeStyle Libre continuous glucose monitors (CGMs) for biometric monitoring. Environmental monitoring requires BOSCH® IoT sensors for air quality and movement detection, TEXAS INSTRUMENTS® mmWave radar sensors for presence detection, and SONY® IMX500 image sensors for visual inferencing. These components communicate with edge computing modules such as the NVIDIA® Jetson Xavier NX or GOOGLE® Coral Edge TPU, which facilitate on-device preprocessing before sending the data to cloud-based inferencing agents.

The Inference Agent Controller (IAC) relies on specialized AI accelerators to manage high-speed machine learning inferencing tasks. The system is optimized to function on NVIDIA® A100 Tensor Core GPUs, GOOGLE® Tensor Processing Units (TPUs), and AMD® Instinct MI210 accelerators for large-scale inferencing tasks. These devices work alongside AI inference frameworks such as TENSORFLOW®, PYTORCH®, and ONNX® Runtime, which allow seamless model deployment and inference execution.

The Rules Controller (RC) is hosted on a secure, high-performance computing cluster that supports real-time decisioning and governance rule management. The preferred setup consists of IBM® Power10 servers, which deliver exceptional AI processing capabilities with integrated AI inferencing cores. Additional rule processing is handled using INTEL XEON® scalable processors within secure server environments such as AWS® Nitro-based EC2 instances or GOOGLE® Cloud Confidential VMs. The RC continuously updates governance logic using a distributed ledger system based on HYPERLEDGER FABRIC® to ensure auditability and compliance.

The Content Control Module (CCM) is responsible for privacy-preserving data handling and secure data governance. The implementation includes HEWLETT PACKARD ENTERPRISE® (HPE) Gen10 secure servers for real-time encryption and anonymization of data, while storage integrity is maintained via WESTERN DIGITAL ULTRASTAR® DC HC560 high-capacity drives with AES-256 encryption. Secure memory environments use INTEL® SGX enclaves and AMD® SEV-SNP technologies to protect data during inference processing. The CCM interacts with privacy-preserving computation frameworks such as GOOGLE® private join and compute and MICROSOFT SEAL® homomorphic encryption to ensure compliance with regulatory standards such as GDPR and HIPAA.

The Action Agents herein execute the system's recommended tasks, requiring high-speed automation controllers and cloud-interfaced IoT components. SIEMENS® SIMATIC S7-1500 PLCs manage industrial automation responses, while RASPBERRY PI 4® and BEAGLEBONE AI-64® facilitate lightweight task execution in consumer applications. For remote actuation, BOSCH® IoT Suite provides a robust framework for edge-to-cloud integration, ensuring seamless execution of automated interventions based on AI inferencing results.

Networking and connectivity within the system require low-latency, high-bandwidth communication to facilitate real-time decisioning. The system leverages CISCO CATALYST 9600 SERIES® switches for high-throughput networking, with ARUBA 9000 SERIES® gateways ensuring secure, zero-trust connectivity between distributed components—this kind of switch is exemplary but generally indicative of the kinds of switches useful in embodiments of the instant application. Edge AI devices operate on WI-FI 6E® and 5G mm Wave® connections, using QUALCOMM SNAPDRAGON X65® modems for ultra-fast data transmission.

A network switch herein is a hardware device that connects multiple devices (such as computers, servers, and IoT components) within a local area network (LAN) and efficiently routes data packets between them. Unlike a hub, which blindly sends data to all ports, a switch operates at Layer 2 (Data Link Layer) or Layer 3 (Network Layer) of the OSI model and intelligently forwards data only to the specific device(s) that need it. Modern switches also support VLANs, QoS (Quality of Service), port mirroring, network security, and traffic shaping.

While CISCO'S CATALYST® 9600 Series is high-end and feature-rich, there are several other enterprise-grade and data center switches from different vendors that are also suitable for AI systems, inference clusters, and high-performance edge networks: ARISTA NETWORKS® switches; JUNIPER NETWORKS® switches; HPE ARUBA® switches; NVIDIA/MELLANOX® switches; and Extreme Networks® switches.

Of particular note for systems and methods herein (i.e., a high-throughput, low-latency environment for system processes herein) include, but are not limited to, the ARISTA 7280R SERIES® (which is optimized for scalable inference clusters); the JUNIPER® QFX5200 (which is optimal for secure, dynamic rule-managed infrastructures); and the NVIDIA SPECTRUM SN3700® especially for use of NVIDIA GPUs throughout an AI architecture.

This hardware ecosystem ensures that the system operates efficiently under various workloads, delivering rapid AI-driven insights while enforcing strict governance protocols. By integrating industry-leading AI accelerators, secure computing environments, and IoT-driven automation, the invention enables an adaptive and privacy-compliant decision-making framework capable of addressing complex real-world challenges in healthcare, compliance, security, and personal wellness.

In a preferred embodiment, the system is implemented as an AI Diabetes Prevention Agent (DPA) designed to help users make healthy lifestyle decisions to prevent or manage early-stage diabetes. This embodiment showcases how the Adaptive AI Governance and Inference System may be configured for health optimization without the need for continuous glucose monitoring (CGM).

The DPA synthesizes various data sources, including user-entered logs, wearable device data, environmental context, and historical behavior patterns. Unlike conventional systems that rely on frequent biometric sensing, the DPA leverages predictive modeling and personalized rule-based governance to identify patterns of risk and intervene proactively. For instance, the system may suggest hydration, dietary alternatives, or physical activity based on current user context and predictive analysis of behavior trends.

This embodiment was validated through a pilot coaching program involving seven participants across diverse social vulnerability indices. Within just three months, 29% met their six-month weight loss goal, while 57% achieved between 50% and 75% of that goal. Physical activity goals were similarly exceeded, with 57% already meeting or surpassing the 150-minute weekly exercise target. These results outperformed benchmarks established by the CDC's Diabetes Prevention Program, which typically sees 35-50% of participants reach target weight loss and 50-60% reach activity goals within 6-12 months.

The system's preemptive functionality was demonstrated through tailored nudges, goal tracking, automated check-ins, and dynamic action plans, all delivered through an interface that responds to evolving user context. These interactions are governed by the Rules Controller, which selects Virtual Experts trained in nutrition, behavioral motivation, and diabetes education. These experts adapt over time based on user input, progress, and real-world evidence collected through the system's inferencing and action-feedback loop.

This real-world demonstration provides a proof of concept for the commercial deployment of the invention. It validates that agentic AI systems governed by domain-specific rule sets and preemptive inferencing can lead to meaningful behavior change, even without costly or continuous biometric sensors.

For clarity, all brand names or specific hardware references included in this application are intended to be illustrative and not limiting. Any hardware, whether current or future, that can perform the claimed functions falls within the scope of the invention. The system is designed to operate flexibly across different platforms, processors, sensors, and connectivity infrastructures, provided they support the modular components and decisioning functions described herein.

The Inference Agent Controller (IAC) utilizes a suite of artificial intelligence (AI) models, which may include supervised learning models, reinforcement learning agents, and unsupervised clustering methods. The inferencing layer supports modular deployment of algorithms such as decision trees, random forests, logistic regression, Bayesian networks, support vector machines (SVMs), and transformer-based deep learning models including BERT and GPT derivatives. Model selection is performed dynamically by the IAC, based on context such as input modality (e.g., image, audio, behavioral log), system latency constraints, available processing resources, and prior model performance in similar contexts. Each model is pre-trained on domain-specific datasets and fine-tuned using anonymized user data through federated learning or local transfer learning techniques.

The system also supports continual and incremental learning. Behavioral outcomes—such as user acceptance of suggestions, biometric feedback, or real-world goal attainment—are logged and periodically used to retrain or recalibrate Inference Agents. This process may occur in the cloud, on edge devices, or via hybrid architecture, using reinforcement learning (e.g., Q-learning or policy gradient methods) or supervised feedback where label data is available.

Each inference engine produces a confidence score and explanation metadata, which are stored and accessible to users or administrators. These metadata elements may include contributing variables, prior-case similarity indices, and associated Virtual Expert justifications. This supports system transparency, auditability, and AI explainability—critical in high-stakes domains such as health or regulatory compliance.

Each Virtual Expert (VE) is a modular, encapsulated logic container composed of one or more of the following: a rule set configuration file, a collection of trained model parameters, inferencing thresholds, recommended action mappings, and dynamic governance bindings. VEs are activated or deactivated by the Rules Controller in response to situational context. Each VE can be versioned, re-trained, or swapped dynamically, enabling continuous evolution of the system's knowledge base.

In some embodiments, VEs may contain embedded graph neural networks, transformer pipelines, or rule engines such as Drools or custom YAML-based interpreters. VEs are interoperable and composable-allowing the system to dynamically assemble new expertise profiles from existing components to handle emerging user needs or external requirements.

To ensure user trust and regulatory compliance, the system provides an optional transparency layer. This layer includes real-time or retrospective explanation generation for any inference or recommendation made by the system. Explanation data may be presented in natural language, graphical formats, or as structured audit logs, depending on user role and access privileges. The system also retains event-based metadata and can generate traceability reports that link decisions to specific models, rule sets, and data inputs, thereby supporting robust auditing and explainability requirements.

### Example I

In one example scenario, a user has configured the system as a health optimization assistant, specifically using the AI Diabetes Prevention Agent embodiment. The system has been preloaded with user preferences, demographic information, behavioral goals, and intermittent biometric readings, such as fasting blood glucose levels and weight measurements. The user does not employ a continuous glucose monitor (CGM).

On a Tuesday morning, the system's Data Collection Interface receives input from a wearable device indicating low overnight physical activity and elevated resting heart rate. Simultaneously, the user enters a breakfast plan via a connected smartphone app, noting an intention to eat pancakes and syrup.

The Content Control Module anonymizes and filters this information, validating it against user privacy settings and health governance parameters. The filtered input is routed to the Inference Agent Controller (IAC), which activates a combination of virtual nutrition and behavior experts (Virtual Experts) governed by the Rules Controller.

The Rules Controller determines, based on CDC prediabetes guidelines and prior user data, that the breakfast may contribute to a postprandial glucose spike. The system generates a confidence score of 89% for this outcome using a transformer-based inferencing engine running on an AI accelerator.

As a result, the Action Agent triggers a proactive intervention. A text notification is sent to the user's smartwatch suggesting alternative breakfast options with a lower glycemic index, such as oatmeal with nuts and cinnamon. It also provides a gentle prompt to schedule a 10-minute walk after eating.

If the user accepts the suggestion, the system logs this outcome and incrementally updates both the user's behavioral model and the priority ruleset for future decisioning. If the suggestion is ignored, the system reduces its confidence in similar future nudges and notes the behavioral deviation.

Throughout the interaction, all data transactions remain compliant with HIPAA and GDPR protocols enforced by the Content Control Module, and a weekly behavior summary is generated for the user's review.

This example illustrates how the system uses multi-source data, rule-based governance, virtual expert orchestration, and preemptive AI inferencing to influence real-world behavior without reliance on continuous biometric sensing.

### Example II

In another exemplary scenario, the system is deployed in the form of a mobile wellness assistant to help a user manage lifestyle choices in socially dynamic environments. The user has configured their Virtual Experts to emphasize blood sugar stability, moderate alcohol intake, and healthy social routines, based on prediabetes risk factors.

On a Friday evening, the system receives contextual inputs from multiple sources. The user's location data, collected via a smartphone GPS module, indicates entry into a restaurant known for rich foods and sugary cocktails. The device's calendar integration suggests the user is attending a friend's birthday dinner. Simultaneously, the user's wearable smartwatch registers an elevated heart rate and light physical activity leading up to the event.

The Content Control Module processes and filters these inputs, applying privacy rules that prevent precise GPS data from being stored. The Inference Agent Controller (IAC) activates a Social Behavior Virtual Expert, trained to evaluate peer-driven contexts. The Rules Controller accesses a library of domain-specific guidelines, including behavioral reinforcement strategies, sugar-alcohol interaction risks, and time-of-day considerations.

The system's inference engine, running on a secure cloud TPU instance, projects a high likelihood of the user exceeding their planned sugar or alcohol intake for the day. With a 92% confidence score, it determines that a preemptive nudge could positively affect decision-making.

The Action Agent sends a personalized, non-intrusive notification to the user's smartwatch: “Hope you're enjoying dinner! Want a tip? A club soda with lime looks just like a cocktail—and helps keep blood sugar steady. Tap for more swaps.”

If the user interacts with the prompt, the system discreetly offers menu suggestions aligned with the user's goals. The user selects one of the recommended dishes and shares it with the table—triggering a positive reinforcement flag in the Rules Controller and strengthening the Virtual Expert's model for future group events.

This interaction highlights the system's ability to make real-time, situation-aware decisions using social, behavioral, and biometric data-all governed by customizable rules and executed with respect for user privacy. No continuous monitoring is required, yet the system delivers timely, beneficial interventions that support sustainable lifestyle change.

In conclusion, the present invention provides a modular, rule-governed AI system capable of delivering real-time, preemptive guidance in dynamic, privacy-sensitive environments. Through the integration of content control, inference orchestration, and governance via Virtual Experts, the system enables proactive, context-aware interventions that support users in making beneficial decisions across a range of domains. These include, but are not limited to, health and wellness, regulatory compliance, personal safety, and behavioral optimization.

A preferred embodiment, the AI Diabetes Prevention Agent, has demonstrated the system's potential to deliver meaningful health outcomes even in the absence of continuous biometric monitoring. This embodiment illustrates the practical utility, adaptability, and user-centered intelligence of the platform. The system's architecture supports real-time situational analysis, dynamic rule application, and secure, privacy-preserving inferencing—all delivered through a scalable framework that accommodates both edge and cloud-based deployments.

By combining deterministic rule logic with adaptive machine learning models and secure data handling, the invention establishes a novel class of AI systems: those capable of autonomous, ethical, and interpretable decision support. This foundational platform empowers users to benefit from AI in a safe, personalized, and extensible manner, advancing the state of the art in applied artificial intelligence.

Any mention of specific brand names, manufacturers, or model numbers (including, but not limited to, microprocessors, graphics processing units, sensors, memory modules, and connectivity hardware) is provided solely for illustrative purposes. Such references are exemplary of the types of hardware components intended to be used in implementing the present invention and are not intended to limit the scope of the invention to any specific brand, model, or manufacturer. Equivalent components, whether now known or later developed, that perform substantially the same function in substantially the same way with substantially the same result are within the scope of this disclosure and the appended claims.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for performing rule-governed, AI-based preemptive decision-making, comprising:
a. a processor configured to execute instructions stored in memory;
b. a data collection interface comprising one or more physical sensors selected from the group consisting of biometric sensors, image sensors, environmental sensors, and wearable monitoring devices;
c. a memory storing rule sets and inferencing models;
d. an Inference Agent Controller (IAC) configured to activate one or more machine learning Inference Agents, each agent executed on a hardware accelerator comprising a graphics processing unit (GPU), tensor processing unit (TPU), or application-specific integrated circuit (ASIC);
e. a Rules Controller (RC) configured to manage and apply one or more governance rules stored in said memory and enforce compliance with privacy or regulatory constraints;
f. a Content Control Module (CCM) configured to filter, encrypt, or anonymize data received from said data collection interface prior to inferencing; and
g. an Action Agent coupled to an output interface or actuator, said Action Agent configured to initiate one or more preemptive tasks based on output from said Inference Agent Controller (IAC) and filtered by said Rules Controller (RC).

2. The system of claim 1, wherein said memory includes configuration data for Virtual Experts, each comprising a modular logic container with rule sets and decision thresholds.

3. The system of claim 1, wherein said Inference Agent Controller (IAC) dynamically selects an inference agent based on input modality and resource availability.

4. The system of claim 1, wherein the Action Agent transmits a recommendation to a user device selected from the group consisting of a smartwatch, smartphone, or tablet.

5. The system of claim 1, wherein said Content Control Module (CCM) applies homomorphic encryption prior to processing by the Inference Agent Controller (IAC).

6. A computing device for preemptive AI-assisted task execution, comprising:
a. a physical housing;
b. a processor within said housing;
c. a non-transitory computer-readable medium storing executable instructions that, when executed by the processor, cause the device to:
i. receive data from one or more onboard or networked sensors;
ii. filter said data based on rule-governed privacy logic;
iii. execute at least one machine learning model on an accelerator device operably connected to said processor to infer contextual state;
iv. evaluate one or more preloaded governance rules against said inferred state; and
v. trigger an output action via a connected user interface or automation actuator.

7. The device of claim 6, wherein said sensor includes a continuous glucose monitor, and said output action comprises a dietary recommendation.

8. A method for executing a preemptive AI-guided task, comprising:
a. receiving, by a computing system, situational input data from one or more sensors;
b. processing said data via a Content Control Module (CCM) to enforce privacy or compliance filtering;
c. activating, by an Inference Agent Controller (IAC), a machine learning inference engine to analyze said filtered input and derive a contextual prediction;
d. applying, by a Rules Controller (RC), one or more domain-specific governance rules to the contextual prediction; and
e. transmitting, by an Action Agent, an intervention signal to a user interface or external system.

9. The method of claim 8, further comprising generating an audit log containing a confidence score, a model identifier, and the applied rule set.

10. The method of claim 8, wherein said governance rules are dynamically updated based on user response or environmental change.
